(19) United States
(12) Patent Application Publication
(10) Pub. No.: US 2026/0248610 A1
(43) Pub. Date: Aug. 27, 2026

(54) Prosthetic Heart Valves Having Leaflet Protection

(71) Applicant: Medtronic, Inc., Minneapolis, MN (US)

(72) Inventors: Justin G. GOSHGARIAN, Minneapolis, MN (US); Yogesh A. DAREKAR, Minneapolis, MN (US); Brenda L. MCINTIRE, Minneapolis, MN (US); Katherine A. MIYASHIRO, Minneapolis, MN (US); Jean-Pierre DUERI, Minneapolis, MN (US); Eric L. PIERCE, Minneapolis, MN (US); Nathan B. WIEMEYER, Minneapolis, MN (US)

(73) Assignee: Medtronic, Inc., Minneapolis, MN (US)

(21) Appl. No.: 18/994,305

(22) PCT Filed: Jul. 12, 2023
(86) PCT No.: PCT/IB2023/057152

§ 371 Date: Jan. 14, 2025

Related U.S. Application Data
(60) Provisional application No. 63/390,586, filed on Jul. 19, 2022.
(60) Provisional application No. 63/416,728, filed on Oct. 17, 2022.

(51) Int. Cl. A61F 2/24

(52) U.S. Cl. CPC A61F 2/2418; A61F 2230/0021

(57) ABSTRACT

Prosthetic heart valves include a stent having an inner surface and an outer surface. A skirt is connected to and spans a circumference of the inner surface. A valve structure is secured to the skirt. The valve structure includes a plurality of leaflets, with adjoining leaflets attached to the skirt next to one another to form commissures. Each leaflet has a free edge that is suspended from the respective commissures associated with the leaflet, with the free edges combining to define an area of coaptation. The valve structure is arranged to define an inflow region opposite an outflow region. The prosthetic heart valve has a compressed arrangement and an expanded arrangement. In various embodiments, the skirt is sized to fully sheathe the leaflets in both the compressed arrangement and the expanded arrangement.

### FIELD

The present technology is generally related to prosthetic heart valves that are delivered in a compressed arrangement via transcatheter procedure. Embodiments are configured to protect the leaflets during compression of the prosthetic heart valve.

### BACKGROUND

A human heart includes four heart valves that determine the pathway of blood flow through the heart: the mitral valve, the tricuspid valve, the aortic valve, and the pulmonary valve. The mitral and tricuspid valves are atrio-ventricular valves, which are between the atria and the ventricles, while the aortic and pulmonary valves are semilunar valves, which are in the arteries leaving the heart. Ideally, native leaflets of a heart valve move apart from each other when the valve is in an open position, and meet or “coapt” when the valve is in a closed position. Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Diseased or otherwise deficient heart valves can be repaired or replaced using a variety of different types of heart valve surgeries. One conventional technique involves an open-heart surgical approach that is conducted under general anesthesia, during which the heart is stopped and blood flow is controlled by a heart-lung bypass machine.

More recently, minimally invasive approaches have been developed to facilitate catheter-based implantation of a prosthetic heart valve or prosthesis on the beating heart, intending to obviate the need for the use of classical sternotomy and cardiopulmonary bypass. In general terms, an expandable prosthetic valve is compressed about or within a catheter, inserted inside a body lumen of the patient, such as the femoral artery, and delivered to a desired location in the heart.

The heart valve prosthesis employed with catheter-based, or transcatheter, procedures generally includes an expandable multi-level frame or stent that supports a valve structure having a plurality of leaflets. The frame can be contracted during percutaneous transluminal delivery, and expanded upon deployment at or within the native valve. One type of valve stent can be initially provided in an expanded or un-crimped condition, then crimped or compressed about a balloon portion of a catheter. The balloon is subsequently inflated to expand and deploy the prosthetic heart valve. With other stented prosthetic heart valve designs, the stent frame is formed to be self-expanding. With these systems, the valved stent is crimped down to a desired size and held in that compressed state within a sheath for transluminal delivery. Retracting the sheath from this valved stent allows the stent to self-expand to a larger diameter, fixating at the native valve site. In more general terms, then, once the prosthetic valve is positioned at the treatment site, for instance within an incompetent native valve, the stent frame structure may be expanded to hold the prosthetic valve firmly in place.

The present disclosure addresses problems and limitations associated with the related art.

### SUMMARY

The techniques of this disclosure generally relate to prosthetic heart valves that are delivered in a compressed arrangement via transcatheter procedure. As delivery devices for transcatheter prosthetic heart valves continue to be reduced in profile, the degree that the prosthetic heart valve will need to be crimped or compressed for loading within the delivery device will correspondingly increase. An increased degree of crimping or compression of the prosthetic heart valve will generally increase the risk and probability that prosthetic heart valve leaflets will be damaged due to the compression. Embodiments are configured to protect leaflets of the prosthetic heart valve during compression of the prosthetic heart valve. Various embodiments are configured such that the leaflets are fully encapsulated, such as by a skirt, seal, or substrate, in both the crimped/loaded arrangement and the expanded, deployed arrangement. The present inventors identified that benefits of the techniques of the disclosure are most noticed as the degree of crimping of the prosthetic heart valve increases.

In one aspect, the present disclosure provides a prosthetic heart valve including a stent frame having an inner surface and an outer surface. A skirt is connected to and spans a circumference of the inner surface of the stent frame. A valve structure is secured to the skirt. The valve structure includes a plurality of leaflets, with regions of adjoining leaflets affixed to the skirt next to one another to form commissures. Each leaflet has a free edge that is suspended from the respective commissures associated with the leaflet, with the free edges combining to define an area of coaptation. The valve structure is arranged to define an inflow region opposite an outflow region. The prosthetic heart valve has a compressed arrangement and an expanded arrangement. In various embodiments, the skirt is sized to fully sheathe the leaflets in both the compressed arrangement and the expanded arrangement.

In another aspect of the present disclosure, in the prosthetic heart valve of any of the previous or subsequent aspects, the edge of the skirt defines at least one of a chevron shape, a square wave shape, or a scalloped shape.

In another aspect of the present disclosure, in the prosthetic heart valve of any of the previous or subsequent aspects, a longitudinal distance from the commissures to the edge of the skirt varies about a circumference of the stent.

In another aspect of the present disclosure, in the prosthetic heart valve of any of the previous or subsequent aspects, a first point along the edge is longitudinally aligned with a first one of the commissures, and second point along the edge is longitudinally aligned with a second one of the commissures, and a third point along the edge is mid-way between the first and second points, and further wherein a longitudinal distance from the third point to the first one of the commissures is greater than the longitudinal distance from the first point to the first one of the commissures.

In another aspect of the present disclosure, in the prosthetic heart valve of any of the previous or subsequent aspects, the skirt extends a first distance beyond the commissures in a direction of the outflow region. In another aspect of the present disclosure, the first distance is between 1 mm and 6 mm.

In another aspect of the present disclosure, in the prosthetic heart valve of any of the previous or subsequent aspects, when the prosthetic heart valve is in the expanded arrangement, the skirt extends a second distance beyond the area of coaptation in a direction of the outflow region. In another aspect of the present disclosure, the second distance is between 1 mm and 6 mm.

In another aspect of the present disclosure, in the prosthetic heart valve of any of the previous or subsequent aspects, when the prosthetic heart valve is in the compressed arrangement, the skirt extends a third distance beyond the free edges in a direction of the outflow region. In another aspect of the present disclosure, the third distance is between 1 mm and 6 mm.

In another aspect of the present disclosure, in the prosthetic heart valve of any of the previous or subsequent aspects, when the prosthetic heart valve is in the expanded arrangement, the skirt extends a distance beyond the area of coaptation in a direction of the outflow region. In another aspect of the present disclosure, the distance is between 1 mm and 6 mm.

In another aspect of the present disclosure, in the prosthetic heart valve of any of the previous or subsequent aspects, when the prosthetic heart valve is in the compressed arrangement, the skirt extends a distance beyond the free edges in a direction of the outflow region. In another aspect of the present disclosure, the distance is between 1 mm and 6 mm.

In another aspect of the present disclosure, in the prosthetic heart valve of any of the previous or subsequent aspects, the skirt spans 360 degrees of the inner surface of the stent.

In another aspect of the present disclosure, in the prosthetic heart valve of any of the previous or subsequent aspects, the prosthetic heart valve is configured such that in transitioning from the expanded arrangement to the compressed arrangement, the free edge of each of the leaflets is forced forward in a direction of the outflow region, and further wherein the skirt is arranged relative to the stent and relative to the valve structure such that an entirety of the free edge of each of the leaflets is within the skirt in the compressed arrangement.

In another aspect of the present disclosure, in the prosthetic heart valve of any of the previous or subsequent aspects, a prosthetic heart valve includes a stent having an inner surface, an outer surface, a first terminal end, and a second terminal end. A skirt is connected to and spans a circumference of the inner surface of the stent. A valve structure is secured to the skirt, the valve structure including a plurality of leaflets, with regions of adjoining leaflets attached to the skirt next to one another to form commissures. Each leaflet has a free edge that is suspended from the respective commissures such that the free edges combine to define an area of coaptation in a closed condition of the valve structure. A skirt extension extends longitudinally between the commissures and the second terminal end of the stent. An arrangement of the valve structure relative to the stent defines an inflow region opposite an outflow region, with the first terminal end of the stent corresponding with the inflow region and the second terminal end corresponding with the outflow region, The prosthetic heart valve has a compressed arrangement and an expanded arrangement.

In another aspect of the present disclosure, in the prosthetic heart valve of any of the previous or subsequent aspects, the skirt extension comprises a plurality of circumferential strips longitudinally spaced apart from each other.

In another aspect of the present disclosure, in the prosthetic heart valve of any of the previous or subsequent aspects, the skirt extension further comprises longitudinal strips circumferentially spaced apart from each other.

In another aspect of the present disclosure, in the prosthetic heart valve of any of the previous or subsequent aspects, the circumferential strips are between 0.05 mm to 1.00 mm in width and are spaced between 0.5 mm to 5.0 mm apart from each other.

In another aspect of the present disclosure, in the prosthetic heart valve of any of the previous or subsequent aspects, the skirt extension extends a distance beyond the commissures in a direction of the outflow region.

In another aspect of the present disclosure, the distance is between 1 mm and 6 mm.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a photograph illustrating a prosthetic heart valve having “severe” leaflet damage.

FIG. 2 is a photograph illustrating a prosthetic heart valve having no leaflet damage.

FIG. 3A is a simplified side view of a prosthetic heart valve in accordance with principles of the present disclosure and in an expanded arrangement.

FIG. 3B is an end view of the prosthetic heart valve of FIG. 3A.

FIG. 3C is a simplified representation of a cross-sectional view of the prosthetic heart valve of FIG. 3A.

FIG. 3D is a simplified representation of a cross-sectional view of the prosthetic heart valve of FIG. 3A in a compressed arrangement.

FIG. 4A is an end view of a portion of the prosthetic heart valve of FIG. 3A in the expanded arrangement and illustrating a leaflet component thereof in an open condition.

FIG. 4B is a cross-sectional view of a portion of the prosthetic heart valve of FIG. 4A, taken along the line 4B-4B.

FIG. 4C is a cross-sectional view of a portion of another prosthetic heart valve in accordance with principles of the present disclosure and akin to the view of FIG. 4B.

FIG. 5A is an end view of the portion of the prosthetic heart valve of FIG. 4A in a compressed arrangement.

FIG. 5B is a cross-sectional view of a portion of the prosthetic heart valve of FIG. 4A, taken along the line 5B-5B.

FIG. 6 is an enlarged, simplified cross-sectional view of a portion of the prosthetic heart valve of FIG. 3A in the compressed arrangement.

FIG. 7 is an enlarged, simplified cross-sectional view of a portion of a conventional prosthetic heart valve in a compressed arrangement akin to the compressed arrangement of FIG. 6.

FIG. 8 is a simplified cross-sectional view of another prosthetic heart valve, with portions illustrated in phantom.

FIG. 9 is a simplified cross-sectional view of a prosthetic heart valve in accordance with principles of the present disclosure, with portions illustrated in phantom.

FIG. 10 is a simplified cross-sectional view of a prosthetic heart valve in accordance with principles of the present disclosure.

FIG. 11 is a simplified cross-sectional view of the prosthetic heart valve of FIG. 3C implanted to a native annulus.

FIG. 12A is a simplified laid open view of an inner surface of a prosthetic heart valve in accordance with principles of the present disclosure.

FIG. 12B is a simplified cross-sectional view of the prosthetic heart valve of FIG. 12A implanted to a native annulus.

FIG. 13 is a simplified laid open view of a prosthetic heart valve in accordance with principles of the present disclosure.

### DETAILED DESCRIPTION

Specific embodiments of the present disclosure are now described with reference to the figures, wherein like reference numbers indicate identical or functionally similar elements.

Aspects of the present disclosure relate to stented prosthetic heart valves (or, more simply, “prosthetic heart valves”) that can be crimped from an expanded arrangement to a compressed arrangement and formatted or configured to minimize potential damage to leaflets of the prosthetic heart valve when forced to the compressed arrangement. The prosthetic heart valves of the present disclosure generally include a stent or stent frame, a skirt, and a valve structure having two or more leaflets. The leaflets are secured to the stent by the skirt. The prosthetic heart valves of the present disclosure can be compressed to a low profile desirable for transcatheter delivery to a target native heart valve. In this compressed, low profile, the skirt protects the leaflets from localized forces, preventing potential damage to the leaflets (e.g., localized imprints). Thus, the prosthetic heart valves of the present disclosure can provide for more uniform loading (e.g., the compressed arrangement) to a delivery device. In other, related embodiments, the prosthetic heart valves can alternatively, or in addition, provide for improved blood velocity on an outflow region thereof when the valve structure is closed, thus decreasing the possibility of clotting.

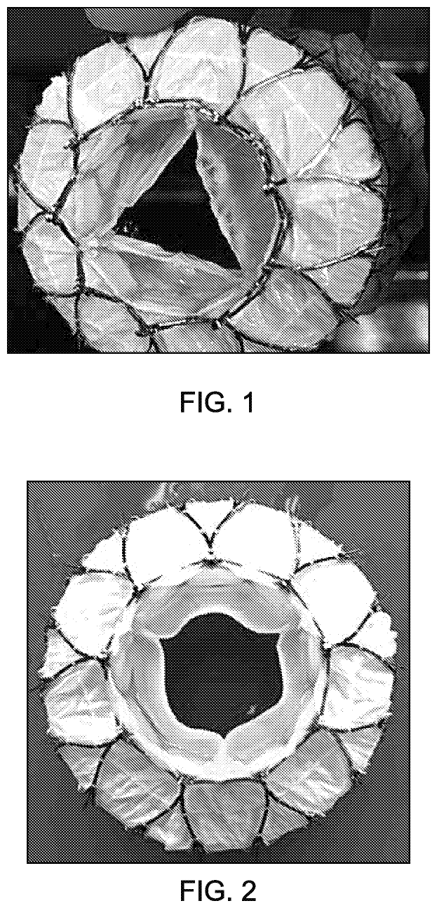

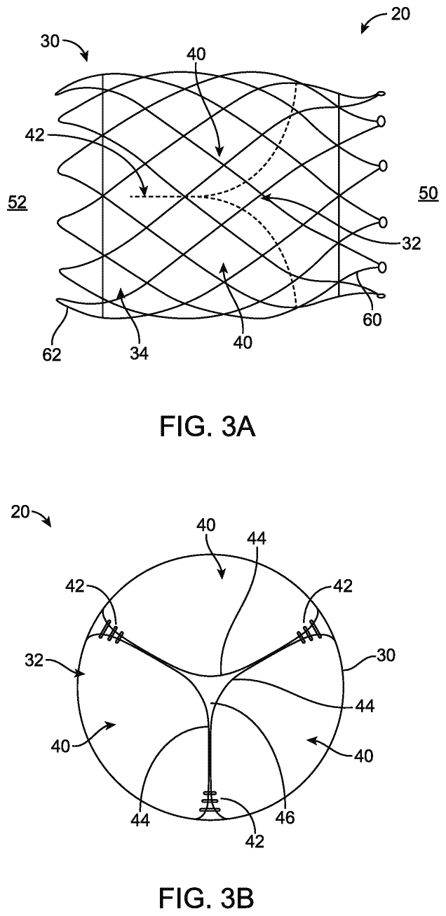

As a point of reference, various techniques and devices are well known for compressing a prosthetic heart valve for loading to a transcatheter delivery device (e.g., self-expanding prosthetic heart valves, balloon-expandable prosthetic heart valves, etc.). Iris-type crimping tools and funnel-type crimping tools two well-accepted examples. Crimping (i.e. compressing) of the stented prosthetic heart valve can damage leaflets and may be observed after the stented prosthetic heart valve is expanded to the expanded arrangement. Leaflet damage can occur in varying degrees. FIG. 1 is an example of severe leaflet damage caused by a crimping process. By way of comparison, FIG. 2 is one example of no damage. Leaflet damage affects the valve structure's ability to coapt and function post deployment. Unplanned creases, indents, thinning, or inelastic stretching of the leaflets can lead to calcification formation in these areas, and damage to leaflet or valve integrity, in vivo. Damaged leaflets are not as durable in vitro. The prosthetic heart valves of the present disclosure can address these crimping-related concerns.

The prosthetic heart valves of the present disclosure can assume a wide variety of configurations appropriate, for example, for implantation at any native valve of the human heart (e.g., mitral valve, aortic valve, tricuspid valve, or pulmonary valve). Thus, the stent, valve structure, and skirt components (as well as any additional, optional components or features) can assume a wide variety of forms. In more general terms, some aspects of the present disclosure relate to arrangements of the skirt relative to the valve structure and/or arrangements of the valve structure relative to the stent. Other than one or both of these attributes, the prosthetic heart valves can have numerous other configurations that may or may not be directly depicted by the drawings.

For example, the stent or stent frame can have various shapes, sizes, and/or other features appropriate for the anatomy of the native heart valve being repaired or replaced and/or connection to a delivery device utilized to deliver the prosthetic heart valve. In general terms, the prosthetic heart valves of the present disclosure include a stent or stent frame carrying a prosthetic valve. The stent or stent frame serves as a support structure, and defines an internal lumen at which the prosthetic valve is maintained. The stents can comprise a number of struts or wire segments arranged relative to each other to provide a desired compressibility and strength to the prosthetic valve. The struts or wire segments are arranged such that they are capable of self-transitioning from, or being forced from (e.g., by a balloon or other expanding structure), a compressed or collapsed arrangement to a normal, radially expanded arrangement. The struts or wire segments can be formed from a shape memory material, such as a nickel titanium alloy (e.g., nitinol). The stent can be laser-cut from a single piece of material, or can be assembled from a number of discrete components. The stented prostheses of the present disclosure may be self-expandable, balloon expandable and/or mechanically expandable or combinations thereof.

Similarly, the valve structure can assume a wide variety of configurations or constructions. For example, valve structures useful with the present disclosure include a bioprosthetic heart valve having tissue leaflets or a synthetic heart valve having polymeric, metallic or tissue-engineered leaflets, and can be specifically configured for replacing valves of the human heart. The valve structure leaflets can be made of pericardial material in some non-limiting examples; however, the valve structure leaflets can instead be made of another material. Natural tissue for the valve body leaflets can be obtained from, for example, mammalian tissue, such as porcine, equine or bovine pericardium. Additionally, synthetic or man-made materials (e.g., polymeric materials, cloths, etc.) can utilized for the valve structure leaflets as is known in the art.

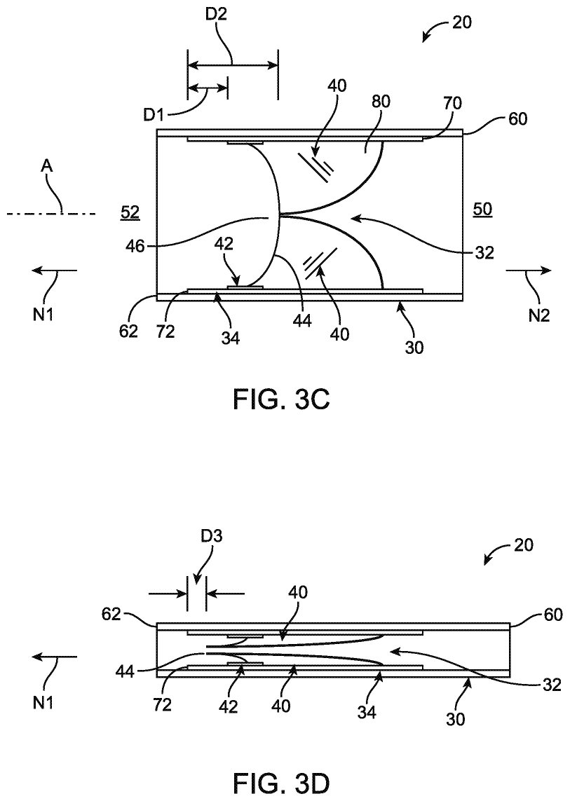

With the above general descriptions of the stent and valve body in mind, one example of a prosthetic heart valve 20 in accordance with principles of the present disclosure is shown in simplified form in FIGS. 3A-3D. The prosthetic heart valve 20 is illustrated herein in order to facilitate description of components attached thereto and/or integrated thereon, and their arrangement relative to other components, to minimize leaflet damage when forced to a compressed arrangement. It will be understood that any number of alternate prosthetic heart valves can be used with the components and/or arrangement to minimize leaflet damage as described herein. As a point of reference, the prosthetic heart valve 20 is transitionable from an expanded arrangement (FIGS. 3A-3C) to a compressed arrangement (FIG. 3D). In some embodiments, the prosthetic heart valve 20 is configured to self-expand from the compressed arrangement to the expanded arrangement; in other embodiments, the prosthetic heart valve 20 is configured to be forced from the compressed arrangement to the expanded arrangement by an auxiliary device (e.g., a balloon as is known in the art). Regardless, the prosthetic valve includes a stent 30, a valve structure 32 (referenced generally in FIG. 3C) and a skirt 34.

The valve structure 32 is attached to the skirt 34 that encloses or is otherwise secured to a portion of an interior or inner surface of the stent 30 (with the inner surface being opposite an outer surface of the stent 30) such that the skirt 34 spans a circumference of the inner surface. The valve structure 32 includes two or more valve leaflets 40 (e.g., while three of the leaflets 40 are depicted in FIG. 3B, in other embodiments, the valve structures of the present disclosure can have only two leaflets, or can have more than three leaflets). The leaflets 40 can be attached along their bases to an interior surface of the skirt 34, for example using sutures or a biocompatible adhesive. Further, adjoining pairs of the leaflets 40 are fastened to the skirt 34 at enlarged lateral edge regions to form commissural joints (or, more simply, “commissures”) 42, with free or unattached edges (sometimes referred to as a “free margin”) 44 of the leaflets 40 forming coaptation edges that meet in an area of coaptation 46 of the valve structure 32, thereby creating a competent seal. The commissures 42 are also secured to the skirt 34, for example above the plane of the area of coaptation 46. An arrangement and configuration of the valve structure 32 is such that the prosthetic heart valve 20 has or defines an inflow region 50 opposite an outflow region 52. In particular, the prosthetic heart valve 20 is constructed to allow blood to flow from the inflow region 50 to the outflow region 52, but prevent blood from flowing in a direction from the outflow region 52 to the inflow region 50.

As a point of reference, in the simplified cross-sectional views of FIGS. 3C and 3D, placement of the commissures 42 and corresponding arrangement of the leaflets 40 are schematically reflected for purposes of generally depicting an operational valve. With the example of FIG. 3B having three of the leaflets 40 and three equidistantly-spaced commissures 42, a cross-sectional plane inclusive of the area of coaptation 46 would not pass through two of the commissures 42 as otherwise implicated by FIGS. 3C and 3D.

An arrangement of the valve structure 32 and the skirt 34 relative to one another and relative to the stent 30 is best seen in FIG. 3C. The skirt 34 in this example extends laterally along an interior of the stent 30, disposed between the valve structure 32 and the stent 30. The skirt 34 and can be formed of a fabric material (e.g., low-porosity woven or knit fabric such as polyester, Dacron fabric, PTFE, etc.) or a natural graft material (e.g., pericardium) that creates a one-way fluid passage when attached to the stent 30. Relationships or arrangements of the valve structure 32 and the skirt 34 can be described with reference to various spatial attributes of the stent 30. For example, the stent 30 can be viewed as defining a longitudinal axis A, and as terminating at a first end 60 opposite a second end 62. In the descriptions below, reference to a “length” is generally measured relative to, or in a direction generally, although perhaps not exactly, parallel to the longitudinal axis A. Thus, a length of the stent 30 can be measured as a distance between the first end 60 and the second end 62. In some embodiments, the first end 60 can be considered or designated as an inflow end of the stent 30, and the second end 62 considered or designated as the outflow end. The skirt 34 defines a first edge 70 opposite a second edge 72, with a length of the skirt 34 traversing the distance between the edges 70, 72. In some embodiments, the first edge 70 can be considered or designated as the inflow edge of the skirt 34, and the second edge 72 considered or designated as the outflow edge. In some embodiments, the first edge 70 of the skirt 34 is more proximate or closer to the first end 60 of the stent 30 as compared to the second end 62; conversely, the second edge 72 of the skirt 34 is more proximate or closer to the second end 60 as compared to the first end 60. While the first and second edges 70, 72 are generally reflected in the embodiments depicted in several of the figures as being uniform (e.g., the first and second edges 70, 72 are each straight or continuous in a single plane perpendicular to the longitudinal axis A), in other embodiments, one or both edges 70, 72 can be non-uniform (e.g., staggered or undulating relative to a single plane perpendicular to the longitudinal axis A). Finally, the valve structure 32 can have a base portion 80 located more proximate or closer to the first end 60 of the stent 30. In at least the expanded arrangement of FIG. 3C, the commissures 42 constitute an opposite extent of the valve structure 32 (e.g., in at least the expanded arrangement of FIG. 3C, the commissures 42 are that portion of the valve structure 32 most proximate or closest to the second end 62 of the stent 30); thus, a length of the valve structure 32 in the expanded arrangement can be measured as a distance between the base portion 80 and the commissures 42. As explained below, in at least the compressed arrangement of FIG. 3D, other portions of the valve structure 32 can be most proximate or closest to the second end 62.

In some embodiments, the skirt 34 is sized and arranged so as to encompass or contain or “sheathe” an entire length of at least the leaflets 40, optionally an entirety of the valve structure 32. For example, a length of the skirt 34 can be greater than a length of the valve structure 32, with opposing extents of the valve structure 32 (e.g., the commissures 42 and the base portion 80) in contact with the skirt 34 (and thus separated from the stent 30 by the skirt 34). In the descriptions below, unless otherwise specified, the term “longitudinally” is reference to a direction generally, although perhaps not exactly, parallel to the longitudinal axis A. In some embodiments, the valve structure 32 and the skirt 34 are arranged such that the skirt 34 extends on the stent 30 longitudinally beyond the commissures 42 by a first distance D1. This relationship can alternatively be described as the second edge 72 of the skirt 34 being located longitudinally between the second end 62 of the stent 30 and the commissures 42, with the second edge 72 being longitudinally spaced from the commissures 42 by the first distance D1. Alternatively, this relationship can be described as the skirt 34 extending longitudinally beyond the commissures 42 in a first direction NI by the first distance D1. The first direction NI can be defined as being generally parallel with the longitudinal axis A and extending toward the second end 62 of the stent 30; further, the first direction NI is opposite a second direction N2 that extends toward the first end 60. In some embodiments, the first distance D1 is about 1 millimeter (mm). In some embodiments, the first distance D1 is between about 1 mm and 2 mm. In some embodiments, the first distance D1 is about 2 mm. In some embodiments, the first distance D1 is greater than about 2 mm and less than about 3 mm. In some embodiments, the first distance D1 is about 3 mm. In some embodiments, the first distance D1 is greater than about 3 mm and less than about 4 mm. In some embodiments, the first distance D1 is about 4 mm. In some embodiments, the first distance D1 is greater than about 4 mm and less than about 5 mm. In some embodiments, the first distance D1 is about 5 mm. In some embodiments, the first distance D1 is greater than about 5 mm and less than about 6 mm. In some embodiments, the first distance D1 is about 6 mm. In some embodiments, the first distance D1 is greater than about 6 mm.

In some embodiments, the valve structure 32 and the skirt 34 are arranged such that in at least the expanded arrangement and with the valve structure 32 closed to block flow (e.g., the arrangement of FIG. 3C), the skirt 34 extends on the stent 30 longitudinally beyond an extent of the area of coaptation 46 by a second distance D2. This relationship can be described as the second edge 72 of the skirt 34 being located longitudinally between the second end 62 of the stent 30 and the area of coaptation 46, with the second edge 72 being spaced from the area of coaptation 46 by the second distance D2. This relationship can also be described as the skirt 34 extending longitudinally beyond the area of coaptation 46 in the first direction N1 by the second distance D2. In some embodiments, the second distance D2 is between about 1 mm and 2 mm. In some embodiments, the second distance D2 is about 2 mm. In some embodiments, the second distance D2 is greater than about 2 mm and less than about 3 mm. In some embodiments, the second distance D2 is about 3 mm. In some embodiments, the second distance D2 is greater than about 3 mm and less than about 4 mm. In some embodiments, the second distance D2 is about 4 mm. In some embodiments, the second distance D2 is greater than about 4 mm and less than about 5 mm. In some embodiments, the second distance D2 is about 5 mm. In some embodiments, the second distance D2 is greater than about 5 mm and less than about 6 mm. In some embodiments, the second distance D2 is about 6 mm. In some embodiments, the second distance D2 is greater than about 6 mm.

As the prosthetic heart valve 20 is forcibly crimped (e.g., radially compressed) from the expanded arrangement to the compressed arrangement, the free edge or margin 44 of one or more (or all) of the leaflets 40 is naturally caused to extend or progress in the first direction N1. In some instances, such as when the prosthetic heart valve 20 is substantively compressed to a low profile for loading to a transcatheter delivery device, the compressed arrangement can include the free edge or margin 44 extending or progressing longitudinally beyond the commissures 42, for example as generally reflected in FIG. 3D.

The inventors have found that one or both the above-described relationships between the skirt 34 and the commissures 42 and/or between the skirt 34 and the area of coaptation 46 promotes substantive compression or crimping of the prosthetic heart valve 20 to the compressed arrangement of FIG. 3D in a manner that protects the leaflets 40. In some embodiments, the prosthetic heart valves of the present disclosure can include the valve structure 32 and the skirt 34 being arranged such that in at least the compressed arrangement (e.g., the arrangement of FIG. 3D), the skirt 34 extends on the stent 30 longitudinally beyond an extent of the free edges or margins 44 by a third distance D3. This relationship can alternatively be described as the second edge 72 of the skirt 34 being located longitudinally between the second end 62 of the stent 30 and an extent of the free edges or margins 44 in the compressed arrangement, with the second edge 72 being spaced from the extent of the free edges or margins 44 by the third distance D3. Alternatively, this relationship can be described as the skirt 34 extending longitudinally beyond the extent of the free edges or margins 44 in the first direction N1 by a third distance D3. In some embodiments, the third distance D3 is between about 1 mm and 2 mm. In some embodiments, the third distance D3 is about 2 mm. In some embodiments, the third distance D3 is greater than about 2 mm and less than about 3 mm. In some embodiments, the third distance D3 is about 3 mm. In some embodiments, the third distance D3 is greater than about 3 mm and less than about 4 mm. In some embodiments, the third distance D3 is about 4 mm. In some embodiments, the third distance D3 is greater than about 4 mm and less than about 5 mm. In some embodiments, the third distance D3 is about 5 mm. In some embodiments, the third distance D3 is greater than about 5 mm and less than about 6 mm. In some embodiments, the third distance D3 is about 6 mm. In some embodiments, the third distance D3 is greater than about 6 mm.

The prosthetic heart valves of the present disclosure optionally include one, two or all of the relationship attributes described above (i.e., the skirt 34 extending laterally beyond the commissures 42 by the distance D1 as described above, the skirt 34 extending longitudinally beyond the area of coaptation 46 in the expanded arrangement by the distance D2, and/or the skirt 34 extending longitudinally beyond the extent of the free edges or margins 44 in the compressed arrangement by the distance D3). Regardless, the inventors find that, when compressed to the compressed arrangement having an outer dimension (e.g., maximum outer diameter) appropriate for a particular transcatheter delivery device, an entirety of each of the leaflets 40, including the free edges or margins 44, is fully contained by, or “inside” of, or sheathed by, the skirt 34. The skirt 34 thus serves to protect the leaflets 40 from potentially damaging contact with the stent 30. Moreover, the above relationship(s) promote a non-damaging interface between the skirt 34 and the leaflet(s) 40 in the compressed arrangement. For example, localized forces at the skirt 34/leaflet 40 are minimized, thus reducing the potential for leaflet imprinting.

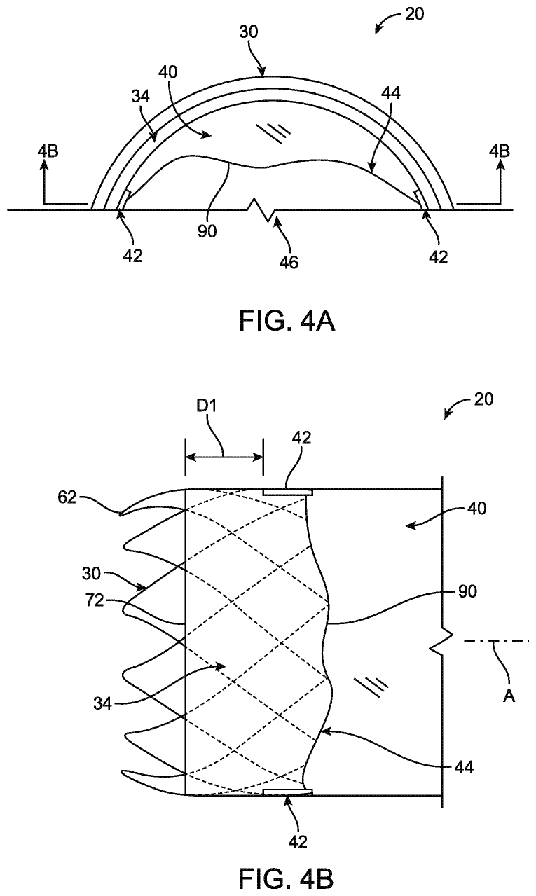

These features can be further understood with reference to FIGS. 4A and 4B. FIG. 4A provides a simplified end view of a portion of the prosthetic heart valve 20, inclusive of a single one of leaflets 40 and the two commissures 42 associated with the leaflet. In the state of FIG. 4A, the prosthetic heart valve 20 is in the enlarged arrangement, and the leaflet 40 is in an open condition (i.e., the free edge 44 is displaced away from the area of coaptation 46, identified generally in FIG. 4A). FIG. 4B is a cross-sectional representation of a portion of the view of FIG. 4A, taken along the line identified at “4B-4B” in FIG. 4A, as viewed from an interior of the prosthetic heart valve 20 (it being recalled that the cross-sectional representations of FIGS. 3C and 3D schematically reflect an arrangement of the leaflets 40; the cross-sectional view of FIG. 4B more accurately reflects the single leaflet 40 relative to the plane 4B-4B of FIG. 4A). Once again, the skirt 34 extends longitudinally beyond the commissures 42 to the second edge 72 by the distance D1.

In some embodiments, this configuration of the skirt 34 can be defined with reference to a mid-region 90 of the free edge 44 (i.e., that region of the free edge 44 approximately equidistantly-spaced from the commissures 42). While opposite ends of the free edge 44 are secured to (and thus constrained by) the stent 30, a remainder of the free edge 44 is free of the stent 30 as thus is unconstrained by the stent 30. As the mid-region 90 is approximately equidistantly spaced from the points or lines of attachment to the stent 30, the mid-region 90 represents the “least constrained” portion of the free edge 44. With this in mind, while the second edge 72 can have a shape that may or may not be uniform in a plane perpendicular to the longitudinal axis A, the distance D1 is established by the second edge 72 along at least a section of the stent 30 that otherwise generally corresponds with the mid-region 90 of the free edge 44. For example, FIG. 4C is a simplified cross-sectional view of an alternative skirt 34′ configuration from a vantage similar to that of FIG. 4B. As shown, the second edge 72′ of the skirt 34′ can have a tapered shape relative to the longitudinal axis A, with the distance D1 as described above being established at a plane corresponding with the mid-region 90. Thus, in some embodiments, a longitudinal distance from the second end 62 of the stent 30 to the second edge 72′ of the skirt 34′ varies about a circumference of the stent 30. In some embodiments, an extent of the second edge 72′ of the skirt 34′ longitudinally beyond the commissures 42 in a direction of the second end 62 of the stent 30 has a minimum distance D4 immediately proximate the commissures 42, and increases to the distance D1 (or maximum extension) at a section otherwise corresponding with the mid-region 90. This relationship can alternatively be described with reference to a first point P1 along of the second edge 72′ that is longitudinally aligned with a first one of the commissures 42, a second point P2 that is longitudinally aligned with a second one of the commissures 42, and a third point P3 that is mid-way between the first and second points P1, P2; with these conventions in mind, a longitudinal distance from the third point P3 to the second end 62 of the stent 30 is less than a longitudinal distance from either of the first and second points P1, P2 to the second end 62. This relationship can alternatively be described as the third point P3 being closer to the second end 62 than either of the first and second points P1, P2. In some embodiments, the second edge 72′ can be described as having a chevron shape (as in the non-limiting example of FIG. 4C). The second edge 72′ can have other varying shapes, such as a square wave shape, a scalloped shape, etc.; in some embodiments, the varying shape is selected so that when the corresponding prosthetic valve is compressed, the second edge 72′ equals the internal circumference of the stent 30.

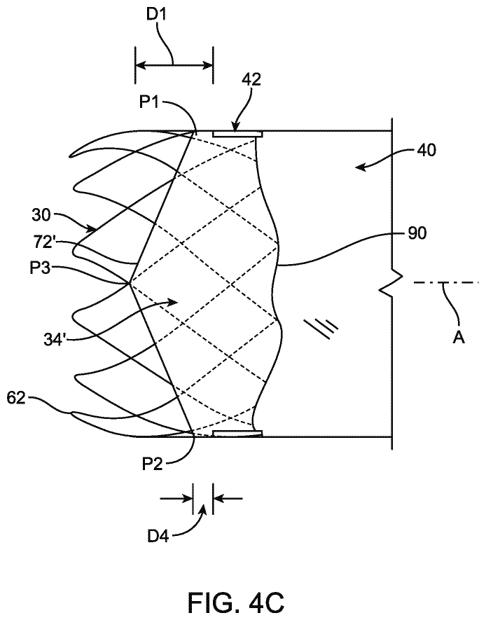

Continuing with the example of FIGS. 4A and 4B, as the prosthetic valve 20 is compressed to a compressed arrangement, the compressive forces can push the unconstrained portion of the leaflet 40 (i.e., portion of the leaflet 40 not otherwise constrained by the commissures 42/stent 30) forward (i.e., in a direction of the second end 62 of the stent 30). FIGS. 5A and 5B provide views akin to those of FIGS. 4A and 4B, but with the prosthetic valve 20 in a compressed arrangement. The free edge 44 is least constrained at the mid-region 90 (as described above), such that forward movement of the free edge 44 in response to compression of the prosthetic valve 30 is more prominent at the mid-region 90, as revealed by a comparison of FIGS. 4B and 5B. However, an arrangement of the skirt 34 and the leaflet 40 relative to the stent 30 is such that even in the compressed arrangement, an entirety of the free edge 44, including the mid-region 90, is within, or is encompassed by, the skirt 34.

The inventors have found that at high compression crimp forces, any portion of the leaflet 40 not otherwise within the skirt 34 can focally stretch, causing a compressed region (or “imprint”) of the leaflet 40 at the interface between the skirt 34 and the unconstrained portion of the leaflet 40; at this junction, the compressive forces can push the unconstrained tissue forward, thinning the tissue locally in that region. The prosthetic heart valves of the present disclosure, for example the prosthetic heart valve 20, overcome these concerns, and can be uniformly crimped and loaded to the delivery device in a manner that reduces or eliminates unplanned creases, thinning or inelastic stretching of the leaflets 40.

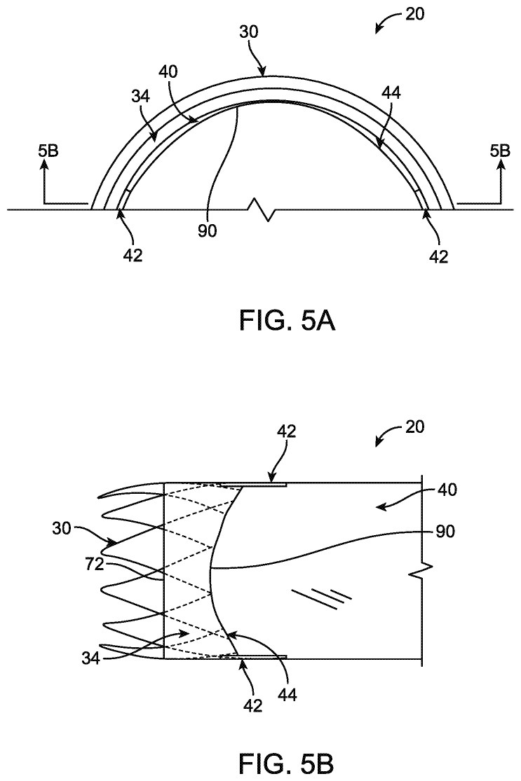

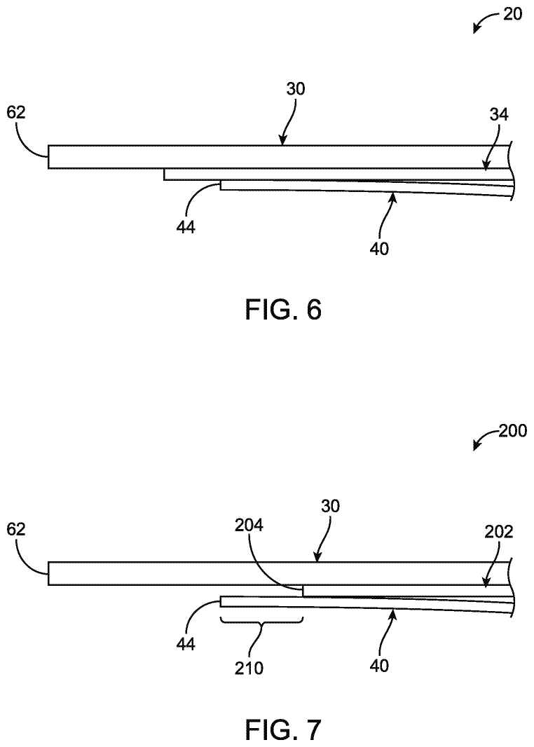

By way of further explanation, FIG. 6 is an enlarged, simplified cross-section view of a portion of the prosthetic heart valve 20 in the compressed arrangement. Commensurate with the above descriptions, the compressive forces have pushed the free edge 44 of the leaflet 40 (i.e., toward the second end 62 of the stent 30). Even in this compressed arrangement, the skirt 34 extends laterally beyond the free edge 44. As a result, the free edge 44, and all portions of the leaflet 40 immediately proximate the free edge 44, contact the skirt 34. By way of comparison, FIG. 7 provides a simplified view of a conventional prosthetic heart valve 200 in a compressed arrangement akin to the view of FIG. 6.

As shown in FIG. 7, the prosthetic heart valve 200 can include a skirt 202 that does not extend longitudinally toward the second end 62 of the stent 30 to the extent described above. Instead, in this prosthetic heart valve 200, a terminal edge 204 of the skirt 202 is located as shown. When the prosthetic heart valve 200 is subjected to the same compressive force applied to the prosthetic heart valve 20 (that otherwise resulted in the arrangement of FIG. 6), the free edge 44 of the leaflet 40 is pushed forward, and extends longitudinally beyond the terminal edge 204 of the skirt 202. This unsupported area of the leaflet 40 is exposed to direct contact with the stent 30 because the skirt 202 does not extend longitudinally beyond the free edge 44. This exposed or unsupported area of the leaflet 40 is generally labeled at 210. The unsupported area 210 can experience thinning and/or wear that leads to thinning, and thus can become damaged (e.g., exhibiting tissue imprinting, unplanned creases, idents or inelastic stretching). As explained herein, the prosthetic heart valves of the present disclosure avoid this issue, and thus present a marked improvement over the conventional prosthetic heart valve 200.

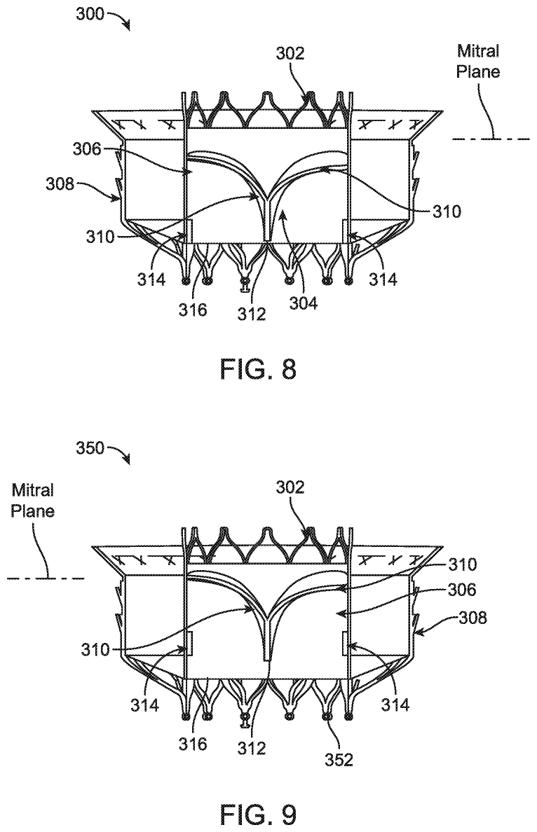

Returning to FIGS. 3A-3D, one of more of the relational attributes described above of the skirt 34 relative to the valve structure 32 can be achieved in various manners. For example, in some non-limiting examples, a conventional prosthetic heart valve design can be reconfigured to add additional skirt material (e.g., adding additional skirt material that extends longitudinally on the stent 30 beyond the commissures 40). Alternatively or in addition, a conventional prosthetic heart valve design can be reconfigured to shift the valve structure 32, and in particular the leaflets 40, relative to the stent 30 and relative to the conventional skirt arrangement along the stent 30. By way of non-limiting example, a known prosthetic heart valve 300 intended for implantation at a native mitral valve is depicted in FIG. 8. The known prosthetic heart valve 300 includes a stent 302, and a valve structure 304 secured to an interior of the stent 302 by a skirt 306. As a point of reference, the known prosthetic heart valve 300 can include various additional features, such as, for example, an outer flange assembly. Regardless of such additional features, the valve structure 304 includes leaflets 310 each forming a free edge or margin that, in a closed condition of the valve structure 304, meet at an area of coaptation 312. Commissures provided with the valve structure 304 are generally indicated at 314. As shown, with the known prosthetic heart valve 300, the skirt 306 does not extend relative to the leaflets 310 to the extent provided with the prosthetic heart valves of the present disclosure. Instead, a terminal edge 316 of the skirt 306 is approximately aligned with the commissures 314.

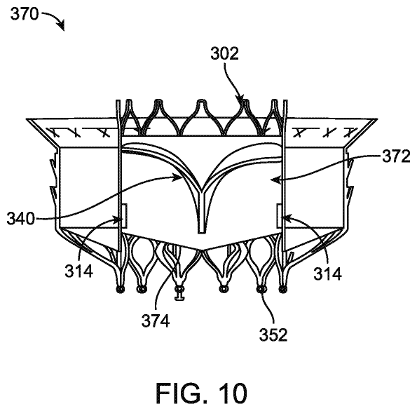

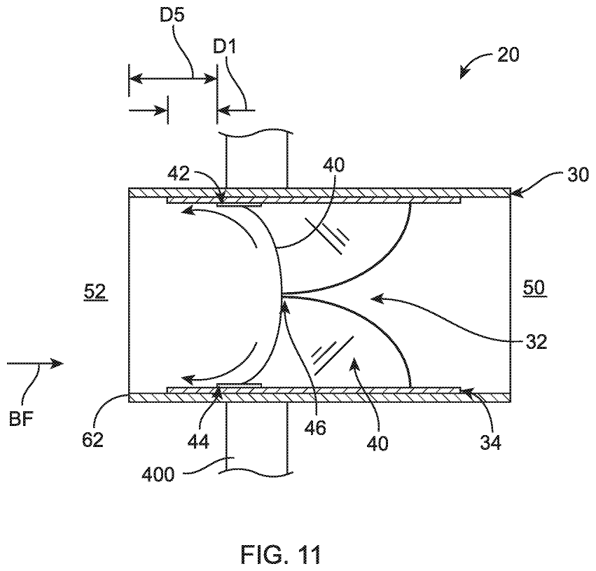

With the above explanations in mind, FIG. 9 depicts a prosthetic heart valve 350 in accordance with principles of the present disclosure. The prosthetic heart valve 350 can be highly similar to the known prosthetic mitral valve 300, for example including the stent 302, the valve structure 304 (not labeled in FIG. 9 for ease of illustration), the skirt 306, and the optional flange assembly 308. However, the prosthetic heart valve 350 of this example incorporates an arrangement of the valve structure 304 relative to the stent 302 and the skirt 306 in accord with the descriptions above and shown, for example, in FIGS. 3A, 3B, 3C, 4B, 4C, and 5B. Relative to the orientation of FIG. 8, the valve structure 304, and in particular the leaflets 310, has been shifted up with the prosthetic heart valve 350 depicted in FIG. 9, as compared to the known prosthetic mitral valve 300. In the context of a prosthetic mitral valve and relative to the line labeled “Mitral Plane”, this shifting can alternatively be considered as “atrialization” of the leaflet position. With the prosthetic heart valve 350 of FIG. 9, the terminal edge 316 of the skirt 306 extends longitudinally beyond the commissures 314 (or commissural plane) and/or longitudinally beyond the area of coaptation 312 commensurate with the descriptions above and thus serves to protect the leaflets 310 from damage during a crimping process. With the optional embodiment of FIG. 9 in which the prosthetic heart valve 350 of that example is configured for implantation at a native mitral valve, the skirt 306 beneficially does not extend all the way to an end 352 of the stent 302 that will otherwise be located near or in the left ventricle upon final implant, as such an arrangement might lead to left ventricular outflow tract obstruction (LVOTO) post implant. FIG. 10 illustrates another prosthetic heart valve 370 in accordance with principles of the present disclosure that is similar to the prosthetic heart valve 350, including the stent 302 and the valve structure 304. The prosthetic heart valve 370 further includes a skirt 372 that can have any of the forms described above. The skirt 372 extends beyond the commissures 314 toward the end 352 of the stent 302. A terminal edge 374 of the skirt 372 can have the tapered or chevron-like shape as shown (and described in greater detail above, for example, with reference to FIG. 4C).

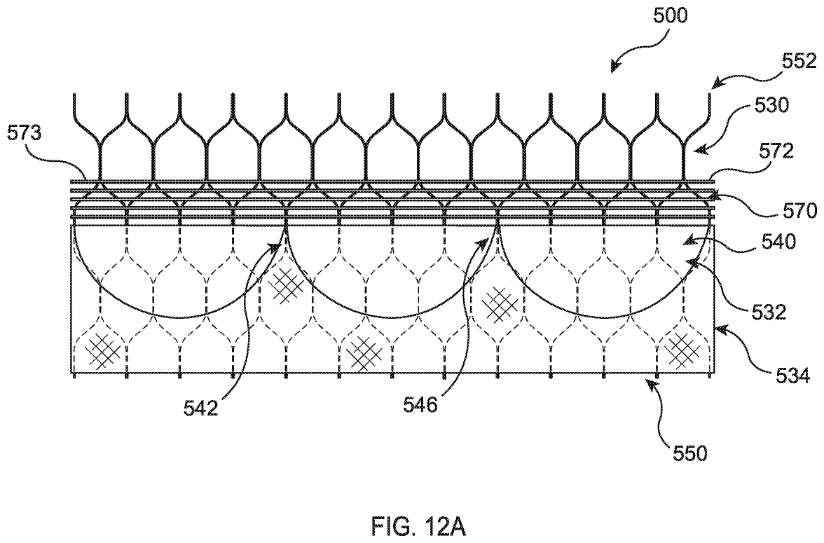

The prosthetic heart valves of the present disclosure can be configured to exhibit features or improvements in addition to, or as an alternative to, the uniform valve loading features described above. For example, the prosthetic heart valves of the present disclosure can be configured to promote a blood velocity on the outflow side of the valve structure when the valve structure is closed that assists in avoiding potential clotting. For example, FIG. 11 is a simplified illustration of the prosthetic heart valve 20 following implantation to a native heart valve annulus region 400 (drawn schematically), and with the valve structure 32 in a closed condition. In the closed condition, the leaflets 40 coapt, preventing blood flow (represented by the arrow “BF”) from passing from the outflow region 52 to the inflow region 50. As described above, the skirt 34 extends longitudinally on the stent 30 in a direction of the outflow region 52 beyond the commissures by the distance D1. At least some of the blood flow BF impinging upon the closed or coapted leaflets 40 is naturally directed in a generally radially outward fashion. Due to the presence of a material of the skirt 34 beyond (i.e., at the outflow region 52) the area of coaptation 46, retrograde blood flow will decrease in between the leaflets 40 and the skirt 34. The decrease in flow is caused as the blood has a longer constricted path to the nadir of the leaflets 40 (as opposed to simply passing through openings in the stent 30 were the skirt material not present). The decrease of blood flow may increase a risk of thrombus formation. With this in mind, in some embodiments, longitudinal extension of the skirt 34 beyond the commissures 42 is limited to the extent necessary to support the free edges 44 in the compressed arrangement (as described above, for example, with respect to FIG. 6), and does not extend to the second or outflow end 62 of the stent 30. In some embodiments, an available distance D5 is defined from the commissures 42 to the second or outflow end 62 of the stent 30; the distance D1 between the second edge 72 of the skirt 34 and the second or outflow end 62 is less than the available distance D5 to minimize impact on retrograde blood flow velocity.

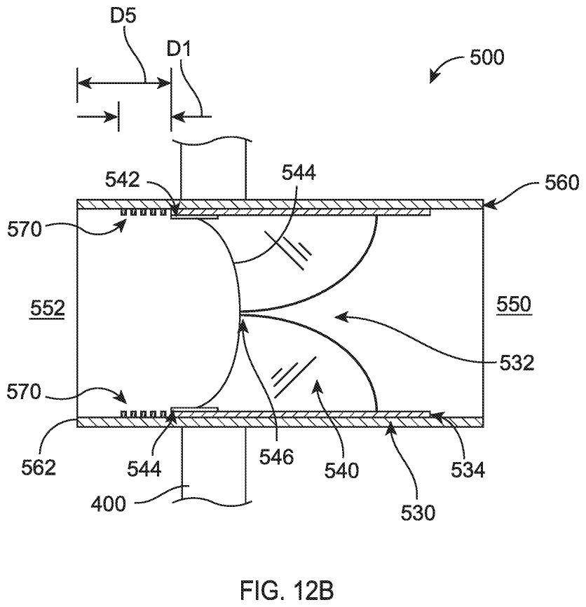

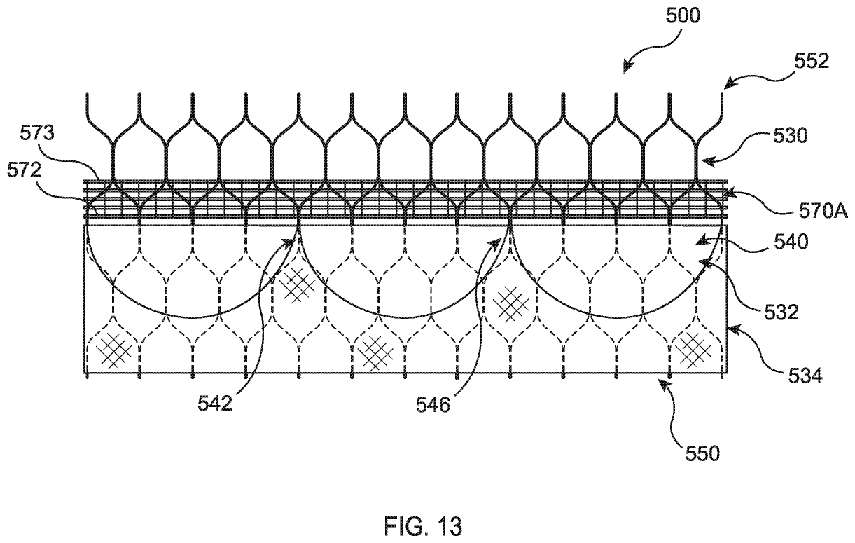

Another embodiment that may optimize longitudinal extension of the skirt while minimizing the impact on retrograde flow velocity is shown with respect to FIGS. 12A-12B and FIG. 13. Generally, the skirt extension is non-continuous. The inventors have found that this non-continuous skirt extension maintains efficacy in preventing leaflet damage during crimping, while minimizing the impact on retrograde flow velocity and minimizing additional skirt material.

In particular, with reference to FIGS. 12A-12B, FIG. 12A shows a prosthetic heart valve 500 in a laid open view showing the inner surface thereof for clarity, while FIG. 12B shows a simplified illustration of the prosthetic heart valve 500 following implantation to a native heart valve annulus region 400. The prosthetic heart valve 500 can be similar to any of the prosthetic heart valves described above, including, but not limited to the prosthetic heart valves 20, or the inner stents of the prosthetic heart valves 350, 370 (including an outer flange assembly).

Similar to the prosthetic heart valves described above, the prosthetic heart valve 500 includes a stent 530, a valve structure 532, a skirt 534, and a skirt extension 570. The details of the stent 530, the valve structure 532, and the skirt 534 will not be repeated here and the descriptions above are incorporated. As described above, the valve structure 532 includes two or more valve leaflets 540 that can be attached along their bases to an interior surface of the skirt 534, for example using sutures or a biocompatible adhesive. Further, adjoining pairs of the leaflets 540 are fastened to the skirt 534 at enlarged lateral edge regions to form commissural joints (or, more simply, “commissures”) 542, with free or unattached edges (sometimes referred to as a “free margin”) 544 of the leaflets 540 forming coaptation edges that meet in an area of coaptation 546 of the valve structure 532. In FIGS. 12A and 12B, the arrangement and configuration of the valve structure 532 is such that the prosthetic heart valve 500 has or defines an inflow region 550 adjacent a first terminal end 560 of the stent 530 opposite an outflow region 552 adjacent a second terminal end 532 of the stent 530. Thus, as described above, the prosthetic heart valve 500 is constructed to allow blood to flow from the inflow region 550 to the outflow region 552, but prevent blood from flowing in a direction from the outflow region 552 to the inflow region 550.

In the embodiments described above, the skirt extends distally (towards the outflow end) beyond the commissures of the leaflets. As described above, this extension can be part of the skirt or a separate piece. In the embodiment of FIGS. 12A-12B, the skirt extension 570, rather than being a continuous extension as shown above, is a non-continuous skirt extension 570. In the embodiment shown in FIGS. 12A-12B, the skirt extension 570 comprises a plurality of thin strips 572 of skirt material. In the embodiment shown, there are five strips 572, but this is not meant to be limiting, and more or fewer strips 572 may be utilized. In an example, the strips 572 may be about 0.05 mm to 1.00 mm in width and may be spaced about 0.5 mm to 5.0 mm apart from each other. However, this is not meant to be limiting, and other widths and spacing may be utilized. In the embodiment of FIGS. 12A-12B, the strips 572 are circumferential, i.e., the strips run in a circumferential direction generally perpendicular to the central longitudinal axis A of the stent 530.

In another embodiment shown in FIG. 13, similar to the embodiment of FIGS. 12A-12B, the skirt extension 570A is non-continuous but includes both circumferential strips 572 and longitudinal strips 574, forming a cross-hatch pattern. The strips 572 and 574 need not be separate strips that are joined together, but can instead be formed as a single piece with both the circumferential strips 572 and the longitudinal strips 574. The longitudinal strips 574 may have a width of about 0.05 mm to 1.00 mm and may be spaced about 0.5 mm to 5.0 mm apart from each other. The circumferential strips 572 may be sized and spaced as described above. Those skilled in the art would recognize that the strips 572, 574 need not be strictly circumferential and longitudinal, as described, but could be at various angles relative to the central longitudinal axis A.

The non-continuous skirt extensions 570 of FIGS. 12A-12B and FIG. 13 maintain the efficacy of the continuous skirt extensions described above because the spacing between the strips 572 is not sufficient to cause leaflet damage, but the spacing enables blood to flow between the strips 572 (and 574) to minimize the impact on retrograde flow velocity. The non-continuous skirt extensions 570 also minimize additional skirt material, thereby positively impacting packing density of the prosthetic heart valve when crimped. The non-continuous skirt extension 570 extends beyond the commissures 542 a first distance D1 as described above. Thus, the first distance D1 between the commissures 542 and an edge 573 of the skirt extension 570 may be between about 1 mm and about 6 mm. In some embodiments, the first distance D1 is between about 1 mm and 2 mm. In some embodiments, the distance is about 2 mm. In some embodiments, the first distance D1 is greater than about 2 mm and less than about 3 mm. In some embodiments, the first distance D1 is about 3 mm. In some embodiments, the first distance D1 is greater than about 3 mm and less than about 4 mm. In some embodiments, the first distance D1 is about 4 mm. In some embodiments, the first distance D1 is greater than about 4 mm and less than about 5 mm. In some embodiments, the first distance D1 is about 5 mm. In some embodiments, the first distance D1 is greater than about 5 mm and less than about 6 mm. In some embodiments, the first distance D1 is about 6 mm. In some embodiments, the first distance D1 is greater than about 6 mm. Further, as described above, in some embodiments, an available distance D5 is defined from the commissures 542 to the second or outflow end 562 of the stent 530; the distance D1 between the edge 573 of the skirt extension 570 and the second or outflow end 562 is less than the available distance D5 to minimize impact on retrograde blood flow velocity.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

1. A prosthetic heart valve (20, 350, 370, 500) comprising:
a stent (30, 302, 530) having an inner surface and an outer surface;
a skirt (34, 306, 372, 534) connected to and spanning a circumference of the inner surface of the stent; and
a valve structure (32, 304, 532) secured to the skirt, the valve structure including a plurality of leaflets (40, 310, 540), with regions of adjoining leaflets attached to the skirt next to one another to form commissures (42, 314, 542, each leaflet having a free edge (44, 544) that is suspended from the respective commissures such that the free edges combine to define an area of coaptation (46, 312, 546) in a closed condition of the valve structure;
wherein an arrangement of the valve structure relative to the stent defines an inflow region (50, 550) opposite an outflow region (52, 552), with opposing terminal ends of the stent including a first end (60, 560) corresponding with the inflow region and a second end (62, 352, 562) corresponding with the outflow region;
wherein the prosthetic heart valve has a compressed arrangement and an expanded arrangement; and
wherein the skirt is sized and arranged to fully sheathe the leaflets in both the compressed arrangement and the expanded arrangement, including an edge (72, 316, 374, 573) of the skirt located longitudinally between the commissures and the second end of the stent.

2. The prosthetic heart valve of claim 1, wherein the edge of the skirt defines at least one of a chevron shape, a square wave shape, or a scalloped shape.

3. The prosthetic heart valve of claim 1, wherein the edge of the skirt defines a varying shape selected such that when the prosthetic heart valve is compressed, the edge covers an inner circumference of the stent.

4. The prosthetic heart valve of claim 1, wherein a longitudinal distance from the commissures to the edge of the skirt varies about a circumference of the stent.

5. The prosthetic heart valve of claim 4, wherein a first point along the edge is longitudinally aligned with a first one of the commissures, and second point along the edge is longitudinally aligned with a second one of the commissures, and a third point along the edge is mid-way between the first and second points, and further wherein a longitudinal distance from the third point to the first one of the commissures is greater than the longitudinal distance from the first point to the first one of the commissures.

6. The prosthetic heart valve of claim 1, wherein the skirt extends a first distance beyond the commissures in a direction of the outflow region.

7. The prosthetic heart valve of claim 6, wherein the first distance is between 1 mm and 6 mm.

8. The prosthetic heart valve of claim 1, wherein when the prosthetic heart valve is in the expanded arrangement, the skirt extends a second distance beyond the area of coaptation in a direction of the outflow region.

9. The prosthetic heart valve of claim 8, wherein the second distance is between 1 mm and 6 mm.

10. The prosthetic heart valve of claim 1, wherein when the prosthetic heart valve is in the compressed arrangement, the skirt extends a third distance beyond the free edges in a direction of the outflow region.

11. The prosthetic heart valve of claim 10, wherein the third distance is between 1 mm and 6 mm.

12. The prosthetic heart valve of claim 1, wherein the skirt spans 360 degrees of the inner surface of the stent.

13. The prosthetic heart valve of claim 1, wherein the prosthetic heart valve is configured such that in transitioning from the expanded arrangement to the compressed arrangement, the free edge of each of the leaflets is forced forward in a direction of the outflow region, and further wherein the skirt is arranged relative to the stent and relative to the valve structure such that an entirety of the free edge of each of the leaflets is within the skirt in the compressed arrangement.

14. The prosthetic heart valve of claim 1, wherein the skirt between the commissures and the edge of the skirt is non-continuous.

15. A prosthetic heart valve (500) comprising:
a stent (530) having an inner surface, an outer surface, a first terminal end (560), and a second terminal end (562);
a skirt (534) connected to and spanning a circumference of the inner surface of the stent;
a valve structure (532) secured to the skirt, the valve structure including a plurality of leaflets, with regions of adjoining leaflets attached to the skirt next to one another to form commissures (542), each leaflet having a free edge that is suspended from the respective commissures such that the free edges combine to define an area of coaptation in a closed condition of the valve structure; and
a skirt extension (570) extending longitudinally between the commissures and the second terminal end of the stent;
wherein an arrangement of the valve structure relative to the stent defines an inflow region opposite an outflow region, with the first terminal end of the stent corresponding with the inflow region and the second terminal end corresponding with the outflow region; and
wherein the prosthetic heart valve has a compressed arrangement and an expanded arrangement.

16. The prosthetic heart valve of claim 15, wherein the skirt extension comprises a plurality of circumferential strips longitudinally spaced apart from each other.

17. The prosthetic heart valve of claim 16, wherein the skirt extension further comprises longitudinal strips circumferentially spaced apart from each other.

18. The prosthetic valve of claim 16, wherein the circumferential strips are between 0.05 mm to 1.00 mm in width and are spaced between 0.5 mm to 5.0 mm apart from each other.

19. The prosthetic heart valve of claim 15, wherein the skirt extension extends a first distance beyond the commissures in a direction of the outflow region.

20. The prosthetic heart valve of claim 19, wherein the first distance is between 1 mm and 6 mm.
